(19) United States
(12) Patent Application Publication
(10) Pub. No.: US 2024/0268857 A1
(43) Pub. Date: Aug. 15, 2024

(54) MYECTOMY DEVICES AND METHODS

(71) Applicant: Mayo Foundation for Medical Education and Research, Rochester, MN (US)

(72) Inventors: Joel L. Kuhlmann, Rochester, MN (US); Nishant Saran, Rochester, MN (US); John M. Angeli, Rochester, MN (US); Joseph A. Dearani, Rochester, MN (US); Hartzell V. Schaff, Rochester, MN (US)

(21) Appl. No.: 18/681,926

(22) PCT Filed: Aug. 16, 2022
(86) PCT No.: PCT/US2022/040416

§ 371 Date: Feb. 7, 2024

Related U.S. Application Data
(60) Provisional application No. 63/234,444, filed on Aug. 18, 2021.

(51) Int. Cl. A61B 17/3207; A61B 17/00

(52) U.S. Cl. CPC A61B 17/3207; A61B 2017/00473

(57) ABSTRACT

This document describes devices and methods for performing septal myectomy procedures in which excess tissue is removed from a thickened, overgrown septum between two ventricles of a heart. For example, this document describes myectomy instruments that can simplify and speed up septal myectomy procedures, and provide enhanced control of the amount of tissue being removed.

### CROSS-REFERENCE TO RELATED APPLICATIONS

This application claims the benefit of U.S. Provisional Application Ser. No. 63/234,444, filed Aug. 18, 2021. The disclosure of the prior application is considered part of (and is incorporated by reference in) the disclosure of this application.

### BACKGROUND

### 1. Technical Field

This document relates to devices and methods for performing septal myectomy procedures in which excess tissue is removed from a thickened, overgrown septum between two ventricles of a heart. For example, this document relates to myectomy instruments that can simplify and speed up septal myectomy procedures, and provide enhanced control of the amount of tissue being removed.

### 2. Background Information

A conventional myectomy procedure is performed through the aortic valve. A standard surgical scalpel is advanced through the aortic valve and is used to slice away the excess tissue. The primary challenges with the current approach are that the surgeon has no real control of the amount of tissue being removed and no objective way to assess how much tissue is being excised.

### SUMMARY

This document describes devices and methods for performing septal myectomy procedures in which excess tissue is removed from a thickened, overgrown septum between two ventricles of a heart. For example, this document describes myectomy instruments that can simplify and speed up septal myectomy procedures, and provide enhanced control of the amount of tissue being removed.

In one implementation, a myectomy instrument includes a handle, a shaft extending distally from the handle, and a cutting blade coupled to a distal end of the shaft. The cutting blade is attached at two attachment portions and has a middle cutting portion extending between the two attachment portions. A proximal-facing edge of the middle cutting portion is sharpened for cutting tissue.

Such a myectomy instrument may optionally include one or more of the following features. The cutting blade may be releasably coupled to the distal end of the shaft. The myectomy instrument may also include a plurality of cutting blades that have differing cutting-depths or cutting-widths. The cutting-depth of the cutting blade may be user adjustable. The cutting-width of the cutting blade may be user adjustable. The cutting blade may be fixedly coupled to the distal end of the shaft. The shaft may be malleable to facilitate user adjustment of the shape of the shaft. The middle cutting portion may be U-shaped.

In another aspect, this disclosure is directed to another myectomy instrument that includes a handle, a shaft extending distally from the handle, and a first interchangeable blade module that is releasably attachable to a distal end of the shaft. The first interchangeable blade module includes a cutting blade attached at two attachment portions and having a middle cutting portion extending between the two attachment portions. A proximal-facing edge of the middle cutting portion is sharpened for cutting tissue. In some embodiments, the myectomy instrument also includes a second interchangeable blade module. The cutting blades of the first and second interchangeable blade module may be configured to facilitate differing cutting-depths or cutting-widths.

In another aspect, this disclosure is directed to a myectomy method that includes advancing any of the myectomy instruments described herein through an aortic valve of a patient, applying the cutting blade to ventricular septal tissue of the patient; and drawing the myectomy instrument proximally such that a strip portion of the ventricular septal tissue is cut by the cutting blade.

Particular embodiments of the subject matter described in this document can be implemented to realize one or more of the following advantages. In some embodiments, the myectomy instruments described herein can be used to remove septal tissue in an advantageously controllable manner and tissue removal amount. For example, in some embodiments the myectomy instruments include a blade that can be used to gradually carve away and remove tissue in strips, as described further below. Such myectomy instruments simplify and speed up septal myectomy procedures, in comparison to conventional procedures that use a scalpel. Moreover, in some embodiments the myectomy instruments described herein can advantageously include adjustable blades or customizable blade cartridges to enable different cutting-widths and/or depths.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of patient's heart that has a thickened ventricular septum.

FIG. 2 is a schematic diagram of the heart of FIG. 1 undergoing a conventional myectomy procedure using a standard surgical scalpel through the aortic valve.

FIG. 3 is a perspective illustration of an example myectomy instrument in accordance with some embodiments provided herein.

FIG. 4 is an exploded view of the myectomy instrument of FIG. 3 showing an interchangeable blade module in accordance with some embodiments.

FIG. 5 illustrates a matrix of example blade modules having differing cutting-depths and widths in accordance with some embodiments provided herein.

FIG. 6 is a perspective illustration of another example myectomy instrument in accordance with some embodiments provided herein.

FIG. 7 is a transverse cross-sectional view of the distal end portion of the myectomy instrument of FIG. 6 taken along cutline 7-7 in FIG. 6.

FIG. 8 is a perspective illustration of another example myectomy instrument in accordance with some embodiments provided herein.

FIG. 9 is an exploded view of the myectomy instrument of FIG. 8.

### Like reference numbers represent corresponding parts throughout.

### DETAILED DESCRIPTION

This document describes devices and methods for performing septal myectomy procedures in which excess tissue is removed from a thickened, overgrown septum between two ventricles of a heart. For example, this document describes myectomy instruments that can simplify and speed up septal myectomy procedures, and provide enhanced control of the amount of tissue being removed.

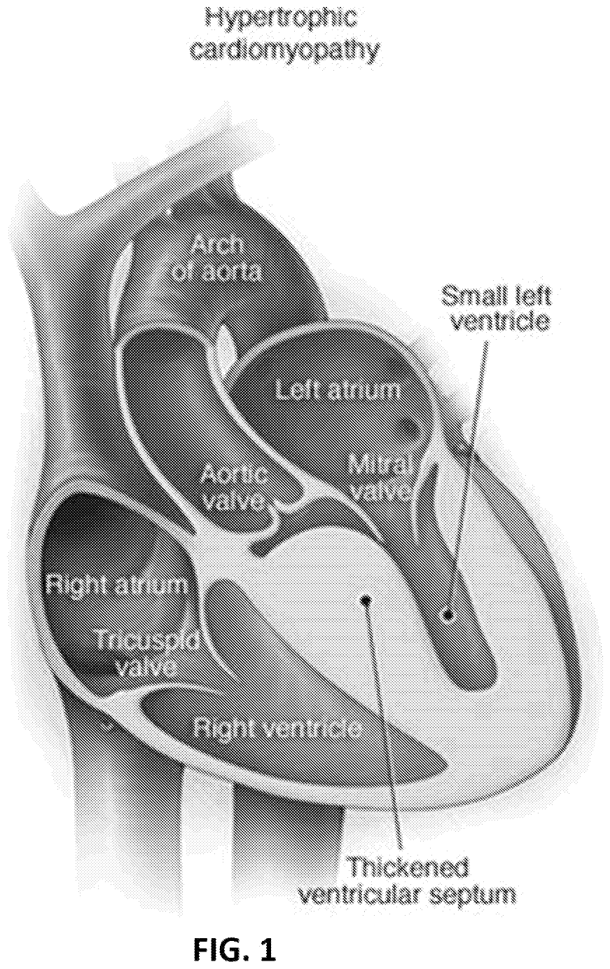

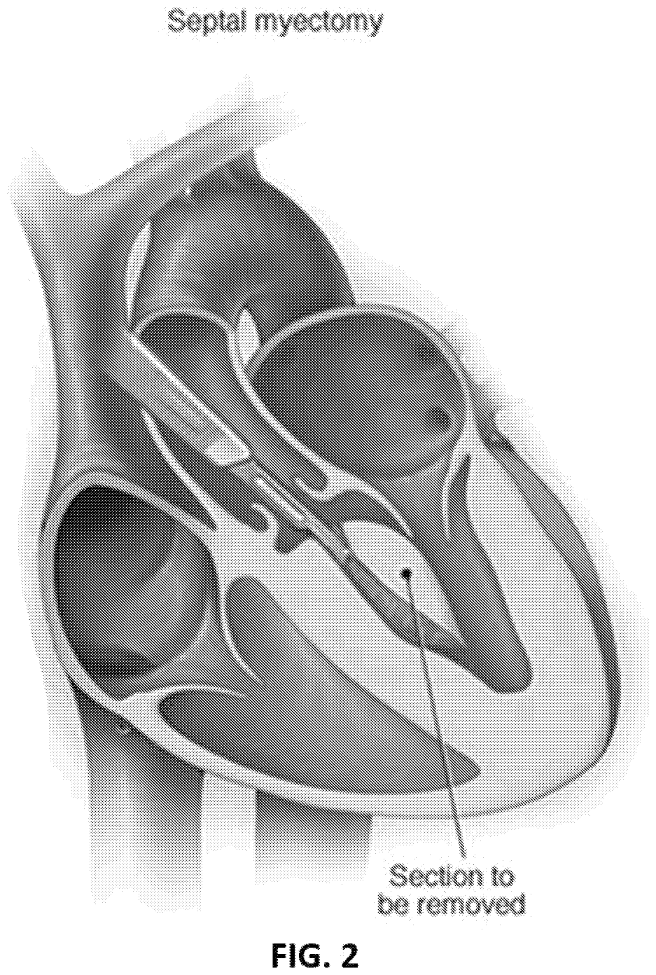

As shown in FIGS. 1 and 2, a conventional septal myectomy procedure is performed through the aortic valve. As shown, a standard surgical scalpel is advanced through the aortic valve and is manipulated by a surgeon to cut away one or more sections of the ventricular septal tissue. One of the challenges with the current approach is that the surgeon has no real control of the amount of tissue being removed.

Accordingly, the inventors have determined that a better approach would be to use a device with a blade that can be inserted through the aortic valve and drawn back (proximal), carving the septal tissue in strips that have a fixed depth and width.

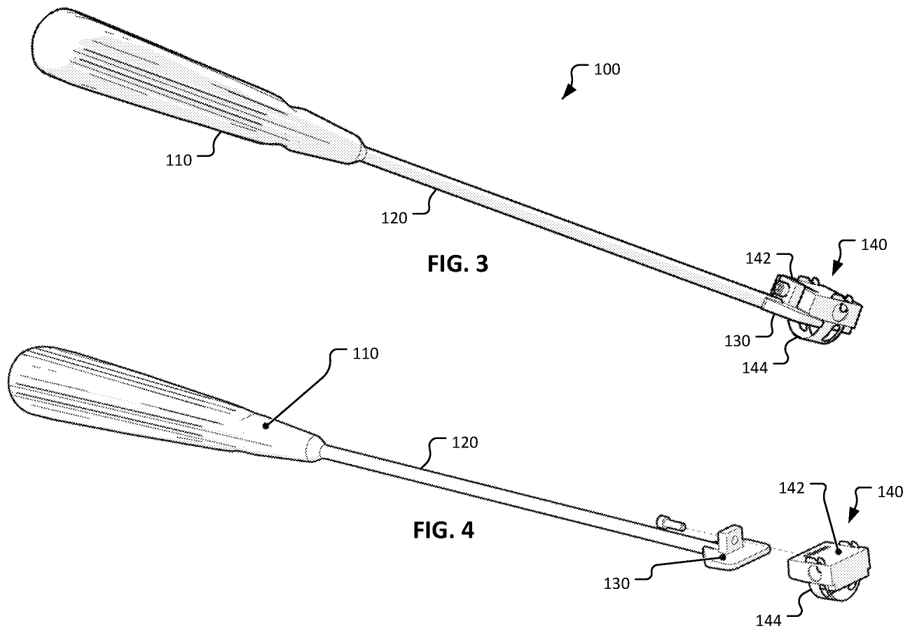

FIGS. 3 and 4 illustrate a first example myectomy instrument 100. The myectomy instrument 100 includes a handle 110, a shaft 120, a blade-mounting portion 130, and a blade assembly 140. The shaft 120 extends distally from the handle 110. The blade-mounting portion 130 is affixed at a distal end of the shaft 120. The blade assembly 140 is releasably attachable to the blade-mounting portion 130.

In some embodiments, the handle 110, the shaft 120, and the blade-mounting portion 130 are reusable (can be sterilized multiple times), whereas the blade assembly 140 is a single-use item. In some embodiments, the entire myectomy instrument 100 is a single-use item.

In some embodiments, the shaft 120 is malleable. That is, the shape of the shaft 120 can be selectively bent into a non-linear configuration (and will remain in the non-linear configuration) as desired by the user. In such a case, the shaft 120 can be bent to have one or more curved portions as desired.

The blade-mounting portion 130 and the blade assembly 140 have cooperative features by which the blade assembly 140 can be easily attached to the blade-mounting portion 130. For example, in some embodiments a single screw can be used to releasably attach the blade assembly 140 to the blade-mounting portion 130. The blade-mounting portion 130 and the blade assembly 140 can also have cooperative features by which the blade assembly 140 is prevented from pivoting in relation to the blade-mounting portion 130. For example, in the depicted embodiment the blade-mounting portion 130 has a flat surface and the blade assembly 140 has an abutting, corresponding flat surface by which the blade assembly 140 is prevented from pivoting in relation to the blade-mounting portion 130.

The blade assembly 140 includes a blade holder 142 and a blade 144. The blade 144 is an elongate blade that has both of its ends attached to the blade holder 142. Accordingly, a U-shaped middle portion of the blade 144 extends between the ends that are attached to the blade holder 142. The U-shaped middle portion of the blade 144 is spaced away from the blade holder 142.

The U-shaped middle portion of the blade 144 is the tissue-cutting portion of the blade 144. The tissue-cutting portion of the blade 144 includes a sharpened proximal edge (facing the handle 110). Accordingly, the blade 144 can cut tissue when the myectomy instrument 100 is drawn proximally.

In the depicted embodiment, a flat bottom surface of the blade-mounting portion 130 is positioned between the U-shaped middle portion of the blade 144 and the blade holder 142 while the blade assembly 140 is attached to the blade-mounting portion 130. In such a case, the open distance between the flat bottom surface of the blade-mounting portion 130 and the U-shaped middle portion of the blade 144 determines the cutting-depth of the myectomy instrument 100. In some embodiments, the blade assembly 140 is attached to the blade-mounting portion 130 in other arrangements by which the open distance between the blade holder 142 and the U-shaped middle portion of the blade 144 determines the cutting-depth of the myectomy instrument 100.

When the U-shaped middle portion of the blade 144 is placed in contact with tissue (e.g., ventricular septal tissue) and the user draws the myectomy instrument 100 proximally with downward pressure, the U-shaped middle portion of the blade 144 cuts the tissue in a strip. The strip of tissue passes through the open space above the U-shaped middle portion of the blade 144.

The blade assembly 140 is readily attachable/detachable from the blade-mounting portion 130. That conveniently allows various types of blade assemblies 140 to be utilized.

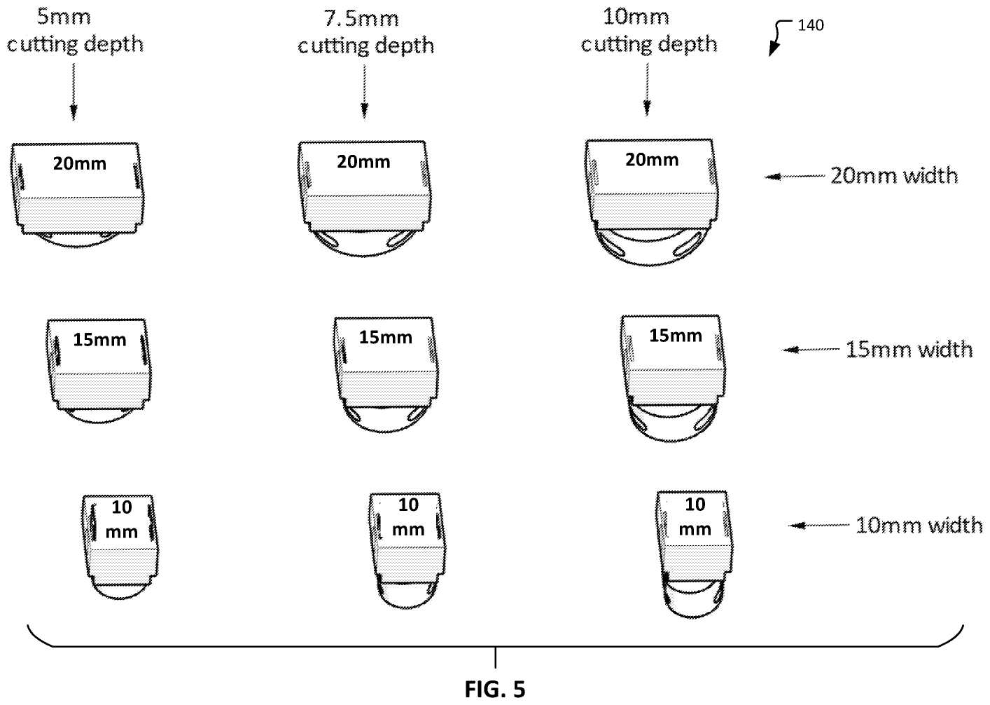

FIG. 5 shows multiple types of example blade assemblies 140. As shown, the blade assemblies 140 can have various widths and various cutting-depths. In the depicted non-limiting examples shown, the cutting-depth can be 5 mm, 7.5 mm, or 10 mm, and the cutting-width can be 10 mm, 15 mm, or 20 mm. These can be combined in any depth and width desired. In addition, these are simply examples and the blade assemblies 140 can be made to have other widths and various cutting-depths.

In some embodiment, the blade assemblies 140 can be insert-molded in a cost-effective manner. That is, a blade can be put into the cavity of an injection mold and molten thermoplastic can be injected to mold it around the blade (resulting in the configurations shown). Other techniques for fabricating the blade assemblies 140 can also be used.

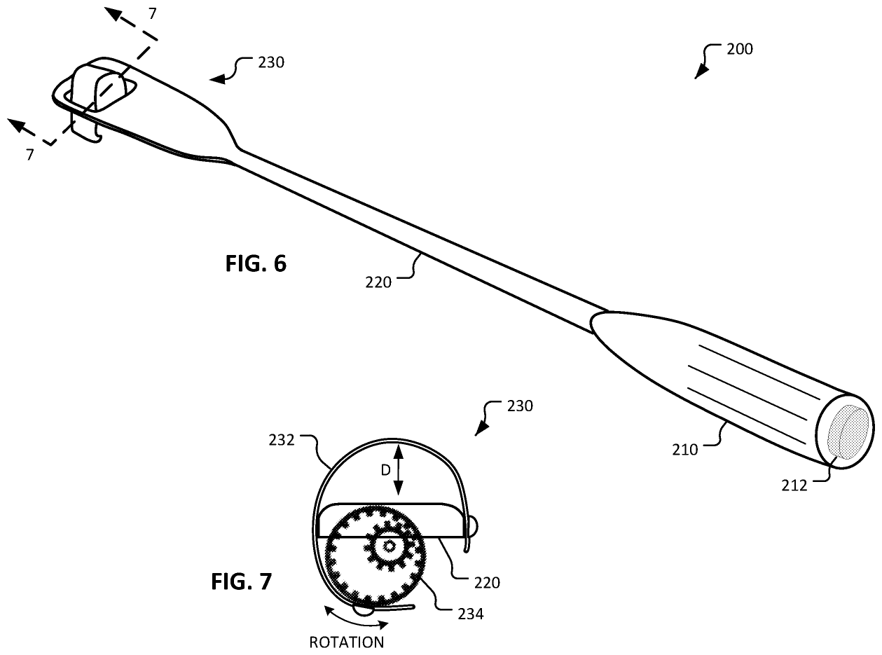

FIGS. 6 and 7 shown a second example myectomy instrument 200. The myectomy instrument 200 includes a handle 210, a shaft 220, and a distal end portion 230. The shaft 220 distally extends from the handle 210. The distal end portion 230 is affixed to the distal end of the shaft 220.

The myectomy instrument 200 can be a single-use or a multiple-use instrument. The shaft 220 can be malleable. The functionality of the myectomy instrument 200 is the same as that of the myectomy instrument 100 in that drawing the myectomy instrument 200 proximally will cut a strip of tissue of a particular width and depth.

The distal end portion 230 includes a blade 232 and an adjustment mechanism 234. The adjustment mechanism 234 is affixed to the blade 232 and movably coupled to the shaft 220. In the depicted embodiment, the adjustment mechanism 234 is a planetary gear assembly (although other mechanisms are also envisioned). Such a gear arrangement can provide for fine control/adjustment of the blade 232.

The adjustment mechanism 234 is controlled by an actuator 212 that is located at the handle. A torsion shaft can extend between the actuator 212 and the adjustment mechanism 234. Accordingly, rotation of the actuator 212 will, in turn, rotate the planetary gear assembly of the adjustment mechanism 234 to cause the cutting-depth “D” of the blade 232 to be adjusted. In some embodiments, the actuator 212 can be locked in a fixed position when the desired cutting-depth “D” has been set. In some embodiments, the handle 210 can include numerical markings that provide an indication of the cutting-depth “D” that has been set by the position of the actuator 212.

In some embodiments of the myectomy instrument 200, the cutting-width is user adjustable. In such a case, a second actuator can be movably coupled to the handle 210. In some embodiments, the second actuator can be movable/slidable along the handle 210 to adjust the cutting-width to be wider or narrower.

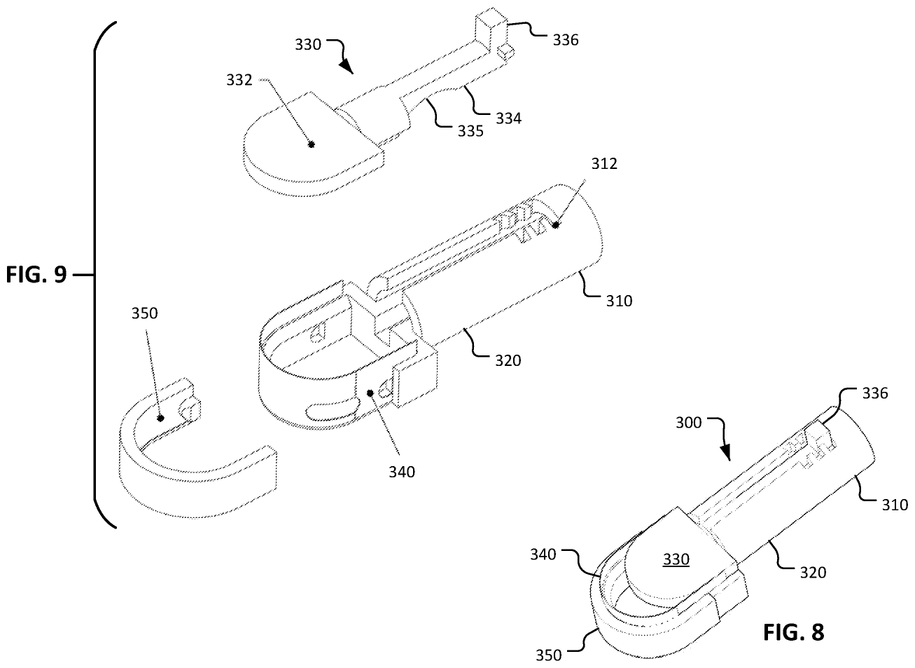

FIGS. 8 and 9 shown a third example myectomy instrument 300. The myectomy instrument 300 includes a handle 310, a shaft 320, a cutting-depth adjustment member 330, a cutting blade 340, and a blade retainer 350. The shaft 320 distally extends from the handle 310. The cutting blade 340 is releasably attached to the distal end of the shaft 320.

The myectomy instrument 300 is not necessarily shown to scale. That is, in some embodiments the shaft 320 is longer than depicted. In some embodiments, an additional shaft can be attached to the proximal end of the handle 310 to lengthen the myectomy instrument 300.

In the depicted embodiment, the sharpened edge of the blade 340 is within a plane that is parallel with the longitudinal axis of the shaft 320. However, in some embodiments the sharpened edge of the blade 340 is within a plane that is non-parallel with the longitudinal axis of the shaft 320. Instead, in some embodiments an angle is defined between the longitudinal axis of the shaft 320 and the plane that the sharpened edge of the blade 340 resides in. The angle can be within a range such as 0° to 30°, or 15° to 45°, or 30° to 60°, or 45° to 75°, or 60° to 90°. In some embodiments, the angle is selectively adjustable by the user.

The cutting-depth of the blade 340 can be adjusted by manipulating the cutting-depth adjustment member 330. Such adjustments will vary the size of the open space defined between the blade 340 and a distal member 332 of the cutting-depth adjustment member 330.

In the depicted embodiment, the cutting-depth adjustment member 330 includes the distal member 332, a shaft 334, and an adjustment member 336.

To change the cutting-depth, the adjustment member 336 can be pushed downward into the handle 310 to elastically deflect the shaft 334 (which has a deflectable portion 335 with a thinned cross-section to facilitate the resilient deflection of the shaft 334 during adjustment). Then, while the adjustment member 336 is being pushed downward, the user can slide the adjustment member 336 distally or proximally to change the size of the open space defined between the blade 340 and the distal end of the cutting-depth adjustment member 330. When the cutting-depth has been set to the desired depth, the user can release the adjustment member 336 and it will snap into engagement with the handle 310 in a latched position. In some embodiments, the handle 310 includes numerical markings that provide an indication of the cutting-depth that has been set by the position of the adjustment member 336.

Other types of cutting-depth adjustment mechanisms are also envisioned. For example, in some embodiments a leadscrew mechanism can be included by which the position of the cutting-depth adjustment member 330 can be moved in relation to the handle 310 and blade 340.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A myectomy instrument comprising:
a handle;
a shaft extending distally from the handle; and
a cutting blade coupled to a distal end of the shaft, the cutting blade attached at two attachment portions and having a middle cutting portion extending between the two attachment portions, wherein a proximal-facing edge of the middle cutting portion is sharpened for cutting tissue.

2. The myectomy instrument of claim 1, wherein the cutting blade is releasably coupled to the distal end of the shaft.

3. The myectomy instrument of claim 1, further comprising a plurality of cutting blades that have differing cutting-depths or cutting-widths.

4. The myectomy instrument of claim 1, wherein the cutting-depth of the cutting blade is user adjustable.

5. The myectomy instrument of claim 1, wherein the cutting-width of the cutting blade is user adjustable.

6. The myectomy instrument of claim 1, wherein the cutting blade is fixedly coupled to the distal end of the shaft.

7. The myectomy instrument of claim 1, wherein the shaft is malleable to facilitate user adjustment of a shape of the shaft.

8. The myectomy instrument of claim 1, wherein middle cutting portion is U-shaped.

9. A myectomy instrument comprising
a handle;
a shaft extending distally from the handle; and
a first interchangeable blade module that is releasably attachable to a distal end of the shaft, wherein the first interchangeable blade module includes a cutting blade attached at two attachment portions and having a middle cutting portion extending between the two attachment portions, wherein a proximal-facing edge of the middle cutting portion is sharpened for cutting tissue.

10. The myectomy instrument of claim 9, further comprising a second interchangeable blade module, wherein the cutting blades of the first and second interchangeable blade module are configured to facilitate differing cutting-depths or cutting-widths.

11. A myectomy method comprising:
advancing the myectomy instrument of any one of claims 1 through 10 through an aortic valve of a patient;
applying the cutting blade to ventricular septal tissue of the patient; and
drawing the myectomy instrument proximally such that a strip portion of the ventricular septal tissue is cut by the cutting blade.

12. The myectomy method of claim 11, further comprising adjusting a cutting-depth of the cutting blade.

13. The myectomy method of claim 11, further comprising adjusting a cutting-width of the cutting blade.

14. The myectomy method of claim 11, further comprising selecting the cutting blade based on a cutting-depth of the cutting blade.

15. The myectomy method of claim 11, wherein the shaft is malleable to facilitate user adjustment of a shape of the shaft, and wherein the method further comprises reshaping the shaft.
